(19) United States
(12) Patent Application Publication
(10) Pub. No.: US 2009/0258371 A1
(43) Pub. Date: Oct. 15, 2009

(54) METHOD OF DETECTING VERY LOW LEVELS OF ANALYTE WITHIN A THIN FILM FLUID SAMPLE CONTAINED IN A THIN THICKNESS CHAMBER

(75) Inventors: Stephen C. Wardlaw, Lyme, CT (US); Robert A. Levine, Guilford, CT (US)

Correspondence Address: O''Shea Getz P.C., 1500 MAIN ST. SUITE 912, SPRINGFIELD, MA, 01115, US

(73) Assignee: Abbott Point of Care, Inc., Princeton, NJ (US)

(21) Appl. No.: 12/417,266

(22) Filed: Apr. 2, 2009

Related U.S. Application Data
(60) Provisional application No. 61/043,571, filed on Apr. 9, 2008.

(51) Int. Cl. G01N 33/53

(52) U.S. Cl. 435/7.1; 977/774

(57) ABSTRACT

A method and apparatus for the detection and quantification of very low levels of a target analyte using an imaging system is provided. In the case of some analytes such as certain hormones, for example TSH, their levels may be as low as several tens of thousands of molecules per micro liter. These extremely low levels can be measured by using the present invention to count the individual molecules of analyte. The invention also has the advantage of being a primary quantitative method, which is one which needs no standardization.

This application claims the benefit of U.S. Provisional Application No. 61/043,571, filed Apr. 9, 2008.

### BACKGROUND OF THE INVENTION

1. Technical Field

This invention relates to a method and apparatus for the detection and quantification of very low levels of a target analyte using an imaging system such as that disclosed in U.S. Pat. No. 6,929,953. In the case of some analytes such as certain hormones, for example TSH, their levels may be as low as several tens of thousands of molecules per micro liter. These extremely low levels can be measured by using the present invention to count the individual molecules of analyte. The invention also has the advantage of being a primary quantitative method, and therefore does not need standardization.

### SUMMARY OF THE INVENTION

The method is for the detection and quantification of a defined target analyte disposed, for example, as a thin film biological fluid sample contained in a thin thickness planar chamber typically from about two microns (2μ) to ten microns (10μ) in thickness. The target analyte has at least two epitopes. The method works by binding single molecules of the defined target analyte to an immobile substrate although binders directed against more than one epitope may be employed in an assay. The substrate has a capture antibody or ligand bound to it. The antibodies or ligands are directed against a first epitope or epitopes of the target analyte, and are operable to immobilize the analyte and prevent its diffusion; i.e., to bind the target analyte to the substrate. The bound target analyte is then detected by use of a labeled probe. The probe contains one or more antibodies or ligands bound to its surface, which antibody or ligand is directed against a second epitope or epitopes of the target analyte.

The first and second type epitopes must be spatially located on the target analytes so that the binding of one epitope does not prevent the binding of the second epitope. The term “antibody” and “ligand” shall refer to any substance capable of binding strongly and specifically to a target epitope and shall include immune globulins, aptimers, and any biological binding agents of similar high binding affinity.

This method is suitable for detecting and identifying any target analyte which has at least two accessible epitopes. An example of such a target analyte is TSH (Thyroid Stimulating Hormone). A biological fluid specimen sample, preferably blood plasma or serum, is introduced into a chamber whose surface area dimensions are chosen to permit the maximal countable number of molecules of the target analyte per unit area of the sample as described below.

The bottom or top surface of the chamber is formed from a plastic sheet to which anti-alpha-TSH antibodies are bound, in an amount in excess of that needed to capture the highest amount of the target analyte that is desired to be measured. The capture antibodies must be bound irrevocably to the immobile substrate so that during the assay, the antibodies do not leave the surface to which they are bound. This area is called the capture area.

The blood plasma or serum sample is added to the chamber, and all of the TSH molecules in the sample will bind to the immobile substrate containing the capture antibodies, thereby immobilizing all of the molecules present in the sample. The thin (typically less then ten microns (10μ)) chamber thickness allows rapid vertical molecular diffusion so that the diffusion between the two layers of the thin chamber occurs rapidly, allowing all the molecules of the analyte to contact the capture antibody surface. Ideally, the plasma, or other biological fluid being examined, should be clear and free of particles such as cells that might interfere with the binding of analyte or the detection of signal in the assay.

Simultaneously, or after a short initial incubation period, fluorescent nanoparticles which are bound to antibodies, such as anti-beta-TSH antibody, which are specific to a second epitope of the analyte, are added to the sample, also in quantity in excess of that needed to bind the maximal number of molecules to be counted. The nanoparticles are preferably ten to 100 nanometers (10 to 100 nm) in diameter consisting of a Europium fluorescent material, or any detectable nanoparticles, such as those called quantum dots or other fluorescent nanoparticles (Sigma Aldrich, St. Louis, Mo., U.S.A. is a supplier). These fluorescent nanoparticles must be sufficiently small and of such density that they will remain in colloidal suspension unless their surface bound antibody becomes attached to an immobilized analyte.

A single fluorescent nanoparticle containing an antibody/ligand directed against the second epitope of the TSH analytes will attach to each TSH molecule that is bound to the substrate. Those fluorescent nanoparticles that are not immobilized by virtue of their attachment to the immobilized analyte will continue to be in colloidal suspension and move due to Brownian motion. To distinguish bound nanoparticles from unbound nanoparticles, the test chamber is imaged under appropriate fluorescent illumination, in the focal plane of the bound particles, after incubation for a period of time which is long enough to give a measurable rise in signal due to the immobile light emitting nanoparticles, as compared to the emission of the moving light emitting nanoparticles which will cause background light due to unbound signal generating nanoparticles. This time of exposure may be adaptively determined by the measuring instrument but limited in its upper extent since it is possible that the areas may have no bound nanoparticles. Those nanoparticles which remain in one location because they are fixed to the substrate will put all of their photons into just a few pixels, while those which “dance” around due to Brownian motion will distribute their brightness over a much larger area, thereby making the detection of the immobile particles possible. A surface area of the chamber which is free of capture antibodies can serve as the control area.

Using this technique, the concentration of nanoparticles in the imaged area should be small enough so that they do not completely overlap and diminish the ability of the sensor to distinguish the immobile particles. The number of individual distinguishable immobile fluorescent particles is therefore equal to the number of molecules of the target analyte contained in the volume of the chamber above or below the capture antibodies within the capture area. Since the volume of the fluid above the control area is relatively small compared to the volume above the immobilized capture antibody or ligand, it may be ignored for purposes of calculating the total volume of the chamber or narrow passage, acting as a diffusion barrier separating the control area from the capture area which may be used to obtain an exact chamber volume over the capture area. Alternatively, an actual impermeable barrier may be employed to separate the capture area from the control area. The maximum number of molecules that may be measured in the contained sample is defined by the capture area of the chamber and the pixel magnification. The concentration of the target analyte will be the number of molecules detected divided by the sample contents in the chamber above the capture area. The volume of the chamber is defined by the known height of the chamber and the area of the sample, which may be defined by the number of pixels within the sample area and the area/pixel magnification factor. Therefore, if the chamber height and magnification are known, the amount of sample volume may also be determined by the instrument performing the analysis. It is necessary that the bound molecules be bound a sufficient distance from each other so that coincidence of signal from the captured labeled nanoparticles avoided. For example, if the fluorescence of a signal contained on a nanoparticle can be detected over an area of 3 to 10 pixels, and the desired image separation of the nanoparticles is at least twice that distance, or about 15 pixels apart, with a magnification yielding an image size of 0.5 microns/pixel, a one square cm of sample area would contain enough resolution for the detection of maximum of about one to two million molecules per chamber. The lower limit of the amount of molecules detected in the chamber is in theory, one, limited of course, by counting statistics. It will be appreciated by one skilled in the art that the thinner the chamber, the greater the discrimination between bound from free labeled target analyte ligand, but the smaller the volume of the sample contained in the chamber. The larger the area of the chamber, the greater the dynamic range, but the longer the time needed to obtain the images of the chamber for analysis.

A 2 cm² chamber, 10 microns (10μ) in height, holding 2 micro liters in the capture area, would be able to detect the presence of a few molecules in this volume. This corresponds to a sensitivity of about 10 attomolar concentration in the source of the sample. If desired, the sensitivity of the apparatus and method could be linearly increased by increasing the volume of the sample by slowly flowing a 10 microliter to 1,000 microliter sample through the thin chamber, thereby capturing most or all of the molecules in that volume. The flow rate would be in the range of about one to several microliters per second. The assay would be done as previously, but the analysis chamber would be placed between the sample holding reservoir and the waste reservoir, and the addition of the detection nanoparticles would not be done until completion of the flow and the results reported per volume that flowed through the chamber. The increased volume sample could be pushed through the sample, although the use of an absorbent material in the collection chamber could automate the flow. The sample would flow both over the capture and control areas.

It is, therefore, an object of this invention to provide a method for quantifying the amount of single molecule target analytes in blood plasma or serum placed in an analysis chamber.

It is an object of this invention to provide a method of the character described which involves capturing the target analyte molecules on a surface of a planar thin film sample chamber having at least one transparent surface and optically highlighting the captured molecules so that they may be photometrically counted.

### DESCRIPTION OF THE DRAWINGS

This and other objects, features and advantages of the present invention will become more apparent in light of the detailed description thereof, as illustrated in the accompanying drawing.

FIG. 1 is a schematic plan view of a portion of a thin film sample test chamber for use in assaying a plasma or serum sample for a target analyte, in this case TSH.

FIG. 2 is a view similar to FIG. 1, but showing the test chamber after it has been filled with the plasma or serum sample and a plurality of fluorescent analyte presence reporters.

FIG. 3 is a view similar to FIG. 2 but showing an electronic image of the test chamber when the latter is being imaged for the presence of the target analyte.

FIG. 4 is a schematic plan view of an alternative embodiment of a thin film sample test chamber assembly which includes a higher volume sample source area, a compound thin film test chamber area, and a higher volume sample reception area.

FIG. 5 is a schematic plan view similar to FIG. 4, but showing the sample being moved through the thin film test chamber area.

FIG. 6 is a schematic plan view similar to FIG. 5, but showing the imaging of the thin film test chamber area after the sample has been moved there through.

### DETAILED DESCRIPTION OF THE INVENTION

Referring now to FIG. 1 there is shown a portion of a thin film test sampling chamber which is denoted generally by the numeral 2. The test sample being assayed in this case is blood plasma or serum and it is being assayed for the presence of TSH (Thyroid Specific Hormone). The chamber 2 has a surface or wall 4 to which a plurality of ligands 6 is affixed. In this case the ligands 6 will be specific to a first surface epitope of the TSH molecules being assayed.

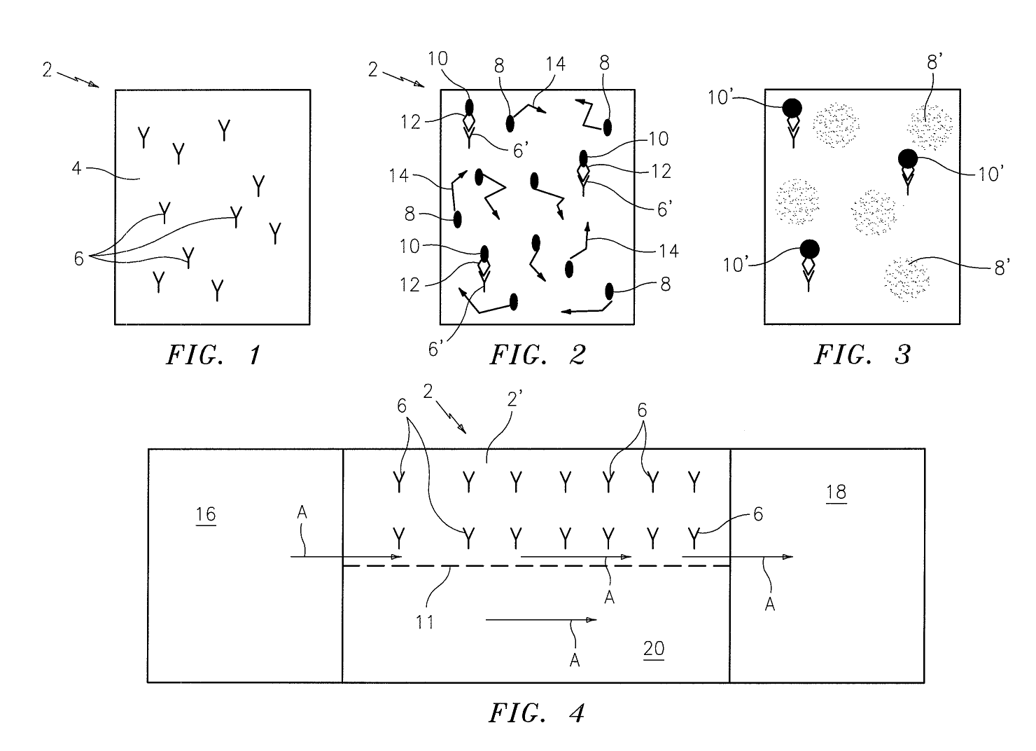

FIG. 2 shows the chamber 2 after it has been filled with a mixture of the plasma being assayed and fluorescent reporter particles 8. The particles 8 include ligands that are specific to a second epitope on the target analyte so that some of the particles will bond with target analyte molecules prior to being placed in the testing chamber 2. Fluorescent reporter particles that bond to the target analyte molecules 12 are designated by the numeral 10. The free unbound fluorescent reporter particles are designated by the numeral 8 in FIG. 2. The target analytes, in this case TSH, are designated by the numeral 12 in FIG. 2. FIG. 2 shows several of the captured analytes 12 and a number of the free unbound fluorescent reporter particles 8. The unbound particles 8 tend to move in the sample 4 as indicated schematically by arrows 14. This being the case, when the sampling chamber 2 is imaged as shown schematically in FIG. 3, the fluorescent signal from the captured reporter particles (on the target analytes) will be relatively bright in the sample, as indicated by the numeral 10′ in FIG. 3, and the fluorescent signal from the free reporter particles will be relatively dim or blurry, as indicated by the numeral 8′ in FIG. 3.

Thus the number of captured target analytes in the sample 4 can be easily determined by imaging the sample 4. Since the volume of the sampling chamber 2 is controlled, the volume of the sample 4 in the chamber 2 is known and the target analyte count can be measured in target analyte/sample volume units.

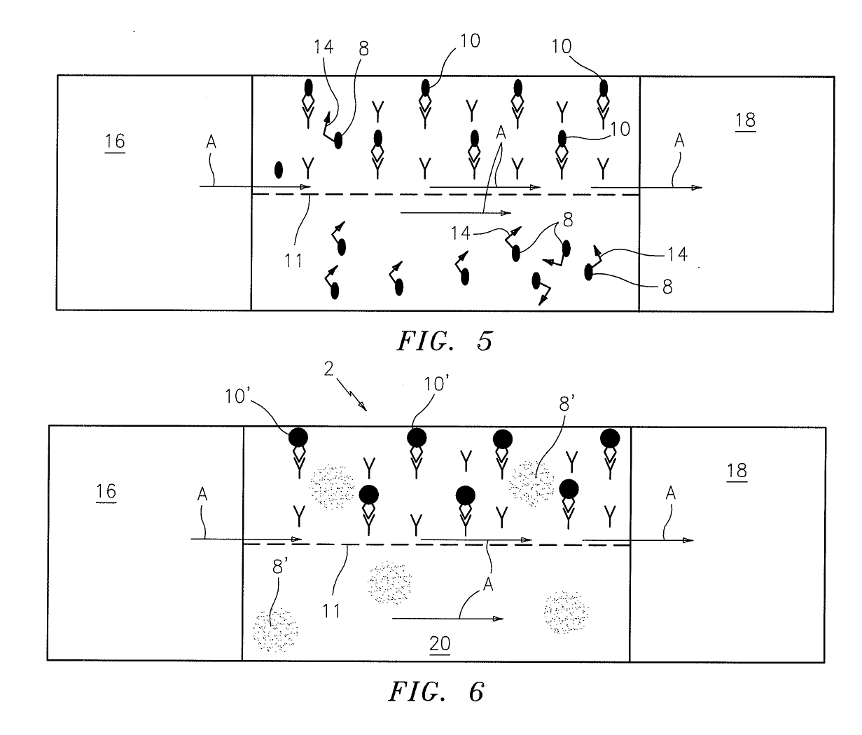

Referring now to FIGS. 4-6, there is shown an embodiment of the device of this invention which is able to sample a larger volume of the sample being assayed. This embodiment includes a sample reservoir 16 in which a larger sample of the plasma or serum to be assayed is placed. The reservoir 16 can hold up to 1 ml, for example, of the sample. The reservoir 16 can have a flexible upper surface which can be depressed so as to compress the sample and pump it through the sample testing chamber component 2 of the assembly. The testing chamber 2 includes a control area 20 which is devoid of capture ligands 6 and the sampling area 2′. This control area is not shown to scale and is much smaller than the capture area or if desired may be connected with a diffusion barrier from the capture area, which includes the analyte capture ligands 6. When the reservoir 16 is compressed, the sample will move in the direction of the arrows A through the sampling area 2′ and the control area 20 at the same time. After passing through the areas 2′ and 20, the sample will be deposited in a reception reservoir 18 which may contain a sample absorbent, if so desired.

FIG. 6 illustrates the image that will be detected in the sample chamber 2′ after the sample has been moved there through. The image will show the bright images 10 of the captured reporter particles, and will show the dimmer and blurrier fluorescent signals 8 from the free or non-captured reporter particles. If the sample test is proven to be valid, then the control area 20 will only include the blurry fluorescent signals 8. The inclusion of the reservoirs 16 and 18 will allow a greater amount of the sample to be assayed, and therefore can provide more valid test results. The broken line 11 in FIGS. 4-6 indicates an impermeable barrier between the sampling area 2′ and the control area 20 which prevents sample crossover between the two areas.

Many modifications of this invention with respect to its construction are possible within the description of the invention. They include the area of the assay chamber ranging from 1 mm² to 400 mm², with a height of 2 microns to 10 microns. The localized bound antibodies are preferably placed in a homogeneous pattern, with the adjacent control area having antibodies with no affinity for the desired analyte, or no antibodies at all. It is the control area that is desirable to assure the absence of, or to control for nonspecific detection of, points of higher intensity that do not correspond to a labeled analyte. It is preferable to limit the diffusion of the sample from the control area to the capture area in order to obtain a more accurate volume determination of the amount of sample that is exposed to the capture antibody. It is also possible, if desired to perform as standard curve where multiple concentrations of known analyte are placed in the analysis chamber and analyzed under similar conditions. The number of detectable discrete signal areas per area imaged in the capture area minus the detectable discrete signals per area imaged in the control area are plotted against the known concentrations of analyte to obtain the standard curve. The results may be used to calculate the concentration of analyte in unknown samples that are analyzed under identical conditions as the standard curve.

Probe signal amplification such as RCAT (rolling circle amplification technology) could be used in place of the nanoparticles since they have the effect of producing localized fluorescent particles.

Since many changes and variations of the disclosed embodiment of the invention may be made without departing from the inventive concept, it is not intended to limit the invention except as required by the appended claims.

What is claimed is:

1. A method for performing an immunoassay of a biological fluid sample for the quantization of a target analyte in a thin film sample chamber, said method comprising the steps of:
providing a plurality of target analyte specific capture antibodies or ligands, sufficient to bind all of the added target analyte, which are fixed to a surface of a thin film sample assay chamber or immobilized structures in the analysis chamber, said capture antibodies or ligands being specific to a first epitope or epitopes on target analyte molecules which are present in said biological fluid sample;
filling said sample assay chamber with a mixture of said biological fluid sample and fluorescent nanoparticles coupled to antibodies that selectively bind to a second epitope or epitopes on target analyte molecules which are present in said biological fluid sample; and
imaging said quiescent sample in said sample assay chamber and counting target analyte molecules which are captured by said immobile capture antibodies and made detectable by imaging the immobilized fluorescent nanoparticles coupled to antibodies that are bound to a second epitope on the immobilized target analyte.

2. The method of claim 1 wherein said nanoparticles are Quantum Dots.

3. The method of claim 1 wherein fluorescent nanoparticles which have become immobilized due to binding to captured target analyte molecules in the sample can be photometrically distinguished from free nanoparticles in the sample as a result of movement of the free nanoparticles due to the Brownian motion phenomenon in the sample.

4. The method of claim 1 wherein the fluorescent nanoparticles are Quantum Dots that have become immobilized due to binding to captured target analyte molecules in the sample and can be photometrically distinguished from free nanoparticles in the sample due to movement of the free nanoparticles resulting from the Brownian motion phenomenon in the sample

5. The method of claim 1 wherein the discrimination between the bound and free labeled detection antibodies is performed by electronic means utilizing an analysis of an image or scan.

6. The method of claim 1 wherein the assayed material is undiluted.

7. The method of claim 1 wherein the number of detectable discrete signal areas per area imaged in the capture is greater than detectable discrete signals imaged per area imaged in the control area and the difference per area multiplied by the area of the capture area is equal to the number of target analyte molecules captured.

8. The method of claim 1 wherein the number of detectable discrete signal areas per area imaged in the capture area is greater than detectable discrete signals per area imaged in the control area and is proportional to the number of target analyte molecules captured in the capture area.

9. The method of claim 1 wherein the number of detectable discrete signals per area imaged in the capture area is greater than detectable discrete signals imaged per area imaged in the control area is indicative of the presence of the target analyte in the sample.

10. The method of claim 1 wherein the chamber contains a control area free of capture antibodies or ligands.

11. The method of claim 1 wherein said nanoparticles are less than about 200 nanometers in diameter.

12. The method of claim 11 wherein said nanoparticles are in the range of about 10 to about 100 nanometers in diameter.

13. The method of claim 1 wherein the sample volume applied is greater than the volume of the analysis chamber.

14. A method for performing an immunoassay of a biological fluid sample for the quantization of a target analyte in a thin film sample chamber, said method comprising the steps of:
providing a supply of a mixture of said biological fluid sample and fluorescent nanoparticles coupled to antibodies that selectively bind to a second epitope or epitopes on target analyte molecules which are present in said biological fluid sample, said supply have a sample capacity which is greater that the sample capacity of said thin film sample chamber;
providing a plurality of target analyte specific capture antibodies or ligands, sufficient to bind all of the target analyte in the supply of said mixture, said antibodies or ligands being fixed to a surface of a thin film sample assay chamber or immobilized structures in the analysis chamber, said capture antibodies or ligands being specific to a first epitope or epitopes on target analyte molecules which are present in said biological fluid sample;
moving said mixture from said supply thereof through said sample assay chamber and into a sample reception reservoir, whereby said target analyte, if present in said sample, will bind to said capture antibodies or ligands in said sample chamber; and
imaging said sample assay chamber and counting target analyte molecules which are captured by said immobile capture antibodies or ligands and made detectable by imaging the immobilized fluorescent nanoparticles coupled to antibodies that are bound to said first epitope on immobilized target analyte.

15. The method of claim 14 wherein said nanoparticles are Quantum Dots.

16. The method of claim 14 wherein fluorescent nanoparticles which have become immobilized due to binding to captured target analyte molecules in the sample can be photometrically distinguished from free nanoparticles in the sample as a result of movement of the free nanoparticles due to the Brownian motion phenomenon in the sample.

17. The method of claim 14 wherein the fluorescent nanoparticles are Quantum Dots that have become immobilized due to binding to captured target analyte molecules in the sample and can be photometrically distinguished from free nanoparticles in the sample due to movement of the free nanoparticles resulting from the Brownian motion phenomenon in the sample

18. The method of claim 14 wherein the discrimination between the bound and free labeled detection antibodies is performed by electronic means utilizing an analysis of an image or scan.

19. The method of claim 14 wherein said nanoparticles are in the range of less than about 200 nanometers in diameter.

20. The method of claim 19 wherein said nanoparticles are in the range of about 10 to about 100 nanometers in diameter.

21. The method of claim 1 wherein the number of detectable discrete signals per area imaged in the capture area is greater than detectable discrete signals per area imaged in the control area compared to a standard curve performed to calibrate the assay chamber in order to determine the concentration of analyte in the sample.
